(19) United States
(12) Patent Application Publication
(10) Pub. No.: US 2009/0170705 A1
(43) Pub. Date: Jul. 2, 2009

(54) PHYLLOSILICATE FORMULATIONS FOR THE CONTROLLED RELEASE OF ACTIVE SUBSTANCES

(75) Inventors: Arno Nennemann, Bergisch Gladbach (DE); Johan Kijlstra, Odenthal (DE); Daniel Rudhardt, Köln (DE); Frank Sicking, Overath (DE)

Correspondence Address: NORRIS, MCLAUGHLIN & MARCUS, PA, 875 THIRD AVENUE, 18TH FLOOR, NEW YORK, NY, 10022, US

(73) Assignee: Bayer Technology Services GmbH, Leverkusen (DE)

(21) Appl. No.: 12/160,688

(22) PCT Filed: Jan. 12, 2007
(86) PCT No.: PCT/EP2007/000235

§ 371 Date: Jul. 11, 2008

(30) Foreign Application Priority Data
Jan. 14, 2006 (DE) 10 2006 001 941.5

(51) Int. Cl. A01N 25/12; A61K 47/00; A61K 8/02

(52) U.S. Cl. 504/367; 514/769; 514/770

(57) ABSTRACT

Novel seed dressing agent formulations comprising of—at least one active agrochemical ingredient for the dressing of seeds, —a dispersion of biologically degradable polyester-polyurethane-polyureas in water whereby the solid content thereof is between 30 and 50 weight %. Said formulation also comprises additives. The invention also relates to a method for the production of a novel seed dressing agent formulation and the utilization thereof for the dressing of seeds.

The present invention relates to pulverulent formulations of active substances, which contain agrochemical, cosmetic, material-protection, veterinary-medical or pharmaceutical active substances and organically modified layered compounds, methods of production thereof and use thereof for the controlled release of the active substances.

The controlled release of active substances is a great challenge for many applications. Fields of application for controlled-release formulations are found in agriculture, cosmetics, medicine and in the area of materials. Depending on the application, various objectives may be important, for example
controlled release of active substances
reduction of toxicity
reduced decomposition of the active substance
reduced volatility of the active substance
reduced washout behavior in soils
reduced odor of the formulation
reduced susceptibility to weathering
easier handling

Phyllosilicates (bentonites, clay minerals) are used as carriers of active substances or as fillers in multicomponent formulations. They can be used as carriers/adsorbents for active substances and other organic molecules on account of the high specific surface and the possibility of organic surface modification. The modification of phyllosilicates and the adsorption of organic molecules on phyllosilicates in general are discussed in a great many works (e.g. Siantar, D. P., Feinberg, B., & Fripiat, J. J., Interaction between organic and inorganic pollutants in the clay interlayer. Clays & Clay Minerals, 42 (1994) 187-96).

Both unmodified phyllosilicates and modified phyllosilicates are used in formulations of active substances. They are also used as a supplement to other constituents of formulations. In combination with polymers, synergistic effects are obtained with respect to the release behavior, as a more or less porous polymer matrix can lead to an additional decrease in release.

Unmodified phyllosilicates are used in pesticide formulations together with various additives and stabilizers. Thus, U.S. Pat. No. 4,304,587 describes for example the use of unmodified phyllosilicates with polymers (polypropylene glycol, polyvinyl alcohol), alcohols (glycol), lactones and other compounds, which serve primarily for altering the form (thickening).

A disadvantage of the unmodified phyllosilicates is their poor capacity for adsorption of hydrophobic active substances.

In order to increase the adsorption of hydrophobic active substances, modified phyllosilicates are used. The modification can be produced for example by ion exchange with inorganic or organic ions. Hermosin, M. C. and Cornejo, J., describe for example improved adsorption of the anionic herbicide 2,4-D on montmorillonite and vermiculite through modification with decylammonium ions (Adsorption of the anionic herbicide 2,4-D on alkylammonium clays. In Proceedings of the 7th Euroclay Conference, ed. M. Störr, K.-H. Henning, and P. Adolphi, Dresden, 1991, pp. 491-5).

This is only given as an example; many studies have been published concerning the adsorption of hydrophobic herbicides on organically modified phyllosilicates.

El Nahhal et al. modified Wyoming montmorillonite with low-molecular aromatic cations such as BTMA and PTMA below the cation exchange capacity of the clay minerals. They found increased adsorption of the hydrophobic herbicides alachlor and metolachlor in comparison with alkylammonium-modified clay minerals. The washout behavior (leaching) and the volatility of the herbicides were reduced (El-Nahhal, Y., Nir, S., Margulies, L., & Rubin, B., Reduction of photodegradation and volatilization of herbicides in organo-clay formulations. Appl. Clay Sci., 14 (1999) 105-19; El-Nahhal, Y., Nir, S., Polubesova, T., Margulies, L., & Rubin, B., Leaching, phytotoxicity and weed control of new formulations of metolachlor. J. Agr. F. Chem. (1997)).

It was also possible to stabilize pesticides against photodegradation. Margulies et al. describe the photostabilization of the active substances by energy transfer to coadsorbed organic cations (Margulies, L., Rozen, H., & Cohen, E., Energy transfer at the surface of clays and protection of pesticides from photodegradation. Nature, 315 (1985) 658-9).

Modification with polyhydroxyaluminum ions (pillared clays) led to a decrease in washout of the herbicide metolachlor compared with the commercial formulation (Nennemann, A., Mishael, Y., Nir, S., Rubin, B., Polubesova, T., Bergaya, F., van Damme, H., & Lagaly, G., Clay-based formulations of metolachlor with reduced leaching. Applied Clay Science, 18, No. 5-6, (2001) 265-75).

Increased adsorption of the pesticide metolachlor was achieved, moreover, with the use of heat-treated bentonites and by coagulation of bentonites with di- and trivalent ions and inclusion in aggregates (Nennemann, A., Kulbach, S., & Lagaly, G., Entrapping pesticides by coagulating smectites. Applied Clay Science, 18, No. 5-6, (2001) 285-98; Bojemueller, E., Nennemann, A., & Lagaly, G., Enhanced pesticide adsorption by thermally modified bentonites. Appl. Clay Sci. 18, (2001), 277ff).

Nir et al. solubilized anionic pesticide in cationic micelles and adsorbed the latter on phyllosilicates. This reduced the washout behavior of anionic herbicides in the soil (Nir, S., Rubin, B., Mishael, Y. G., Undabeytia, T., Rabinovitch, O., & Polubesova, T. Controlled-release formulations of anionic herbicides. [WO 2002052939]. 2002.).

The cited prior art focuses on reducing the release by rain and preventing the washout of the formulation. The above works do not report any results regarding deliberately influencing the amounts of active substance released as a function of time. The washout behavior is determined by spraying vertically positioned soil columns, equilibration over a constant period of time and subsequent detection of the depth of penetration of the active substance in the soil by means of bioassays. In the case of photolytic degradation, a similar procedure is followed, after first treating the formulations with UV light.

Correspondingly produced formulations showed, in time-dependent measurements, the familiar hyperbolic release behavior with high initial rate of release and declining release as time passed. Disadvantages of this release behavior are initially increased phytotoxicity and inadequate efficacy as release continues.

The synergistic effect in the release behavior with a polymer matrix is utilized in some applications. So-called clay-polymer nanocomposites represent one way of employing this synergistic effect. Polymer-clay nanocomposites can be produced for example by interlamellar polymerization, solution or by compounding. The following approaches may be mentioned as examples:

Tsipursky et al. prepared matrix formulations by incorporating phyllosilicates in the polymer matrix by solution or melting (Tsipursky, S. J., Beall, G. W., & Vinokour, E. I. Intercalates and exfoliates formed with N-alkenyl amides and/or acrylate-functional pyrrolidone and allylic monomers, oligomers and copolymers and composite materials containing same. [U.S. Pat. No. 5,849,830]. 1998.).

U.S. Pat. No. 5,160,529 describes interlamellar polymerization for the encapsulation of pesticides. Phyllosilicates were mixed with polyol and polyisocyanate and reaction to the polyurethane was carried out. As a result, a permeable polymer shell was formed, which contained the pesticide.

In US2004-0231231 A, active substance is adsorbed on phyllosilicate, this complex is mixed with dissolved polymer and a controlled-release formulation is produced with a long-term barrier for release of the active substance, for example for an attract-kill application.

Disadvantages of the last-mentioned formulations based on combining modified phyllosilicates with polymers is the increased price because of an additional formulation step and moreover a dilution effect by the polymer matrix, which decreases the formulated amount of active substance.

According to the current state of the art it is known that modified phyllosilicates delay the release of active substances. A release profile from these phyllosilicate formulations is determined by adsorption and desorption phenomena on the phyllosilicate carriers and by diffusion of the active substance from the space between the layers. A disadvantage of these systems is, however, that the rate of release is not controllable. Initially, much more active substance is released in unit time, but then the rate of release decreases continuously (hyperbolic variation). This does not provide uniform supply, for example of a plant, with constant amounts of active substance over a specified period of time. Another disadvantage of this release behavior is an initially increased risk of phytotoxicity and inadequate efficacy as release continues.

Based on the known prior art, the problem is therefore to provide layered-compound formulations of active substances which not only delay the release of active substances even more, but also give a controllable release profile with continuous release of active substance.

This problem is solved with the powder formulations according to the invention and the method of production thereof according to the independent claims, with the dependent claims presenting preferred embodiments of the invention.

The invention therefore relates to powder formulations for controlled, delayed release of active substances containing
at least two organically modified, inorganic layered compounds, each containing at least one active substance and
optionally additives
   characterized in that they can be obtained by a method of production in which
in each case an organically modified layered compound is dispersed in a separate solution of one or more active substances in a solvent or a solvent mixture,
wherein
either the respective solvents or solvent mixtures differ from one another or
the organically modified inorganic layered compounds differ in at least one modifier or, if the modifiers are the same, these differ in their proportion in the composition or
the unmodified inorganic layered compounds forming the basis of the at least two organically modified inorganic layered compounds are different and
the respective solvent or solvent mixture is then removed,
the powder of organically modified layered compound and active substance resulting in each case is homogenized and
the powders resulting in each case from the different solvent or solvent-mixture preparations are mixed together.

The use of different solvents and solvent mixtures in otherwise identical production steps or of organically modified inorganic layered compounds differing in at least one modifier or—if the modifiers are the same—differing in the proportion of the modifiers in the composition in otherwise identical conditions or of the at least two organically modified, inorganic layered compounds differing in the unmodified inorganic layered compounds on which they are based, in otherwise identical conditions, gives rise to a different release behavior. A mixture of such formulations from individual formulations produced with different solvents displays a slower, continuous release, the profile of which can be controlled in a surprisingly simple manner by means of the composition of the mixture.

A preferred object of the invention is, moreover, powder formulations for controlled, delayed release of active substances containing
at least two organically modified, inorganic layered compounds, each containing at least one active substance and
optionally additives
   characterized in that they can be obtained by a method of production in which
in each case an organically modified layered compound is dispersed in a separate solution of at least one active substance in a solvent or a solvent mixture,
wherein the respective solvents or solvent mixtures differ from one another and
the respective solvent or solvent mixture is then removed,
in each case the resultant powder of organically modified layered compound and active substance is homogenized and
the powders resulting in each case from the different solvent or solvent-mixture preparations are mixed together.

The use of different solvents and solvent mixtures in otherwise identical production steps gives rise, surprisingly, to a different release behavior with otherwise similar organically modified inorganic layered compound and similar inorganic layered compounds on which the organically modified layered compounds are based in the layered compound formulation resulting in each case and a mixture of such formulations from individual formulations produced with different solvents displays a slower, continuous release, the profile of which can be controlled by means of the composition of the mixture.

The invention further relates to powder formulations for controlled, delayed release of active substances containing
at least two organically modified, inorganic layered compounds, each containing at least one active substance and
optionally additives
   characterized in that they can be obtained by a method of production in which
in each case an organically modified layered compound is dispersed in an individual solution of one or more active substances in a solvent or a solvent mixture,
wherein the organically modified, inorganic layered compounds each have one or more modifiers and the layered compounds differ in at least one modifier or, if the modifiers are the same, these differ in their proportion in the composition and
the respective solvent or solvent mixture is then removed,
the powder of organically modified layered compound and active substance resulting in each case is homogenized and
the powders resulting in each case from the different solvent or solvent-mixture preparations are mixed together.

The use of such a powder formulation also gives rise to a different release behavior. The at least one different modifier of the respective organically modified inorganic layered compounds or, if the modifiers are the same, the modifiers differing in the proportion in the composition in the layered compound formulation resulting in each case with otherwise identical production steps in the same solvents and solvent mixtures and with the same inorganic layered compounds forming the basis of the organically modified layered compounds, makes it possible, in the powder formulations according to the invention obtainable from them, to have an influence, in a surprisingly simple manner, on continuous release, the profile of which can be controlled by means of the composition of the mixture.

The invention further relates to powder formulations for controlled, delayed release of active substances containing
at least two organically modified, inorganic layered compounds, each containing at least one active substance and
optionally additives
   characterized in that they can be obtained by a method of production in which
in each case an organically modified layered compound is dispersed in an individual solution of one or more active substances in a solvent or a solvent mixture
wherein the unmodified inorganic layered compounds forming the basis of the at least two organically modified, inorganic layered compounds are different and
the respective solvent or solvent mixture is then removed,
the powder of organically modified layered compound and active substance resulting in each case is homogenized and
the powders resulting in each case from the different solvent or solvent-mixture preparations are mixed together.

The use of different unmodified inorganic layered compounds forming the basis of the at least two organically modified, inorganic layered compounds in otherwise identical production steps in the same solvents or solvent mixtures and with organically modified inorganic layered compounds provided with the same modifiers in the same composition gives rise to a different release behavior in the layered compound formulation resulting in each case and a mixture of such formulations from individual formulations produced with different inorganic layered compounds displays a slower, continuous release, the profile of which can be controlled in a surprisingly simple manner by means of the composition of the mixture.

The invention further relates to any mixtures of the preferred powder formulations described above.

The powder formulations according to the invention have the advantage that, owing to the controllable release profile, supply with active substance takes place continuously over a longer period of time, washout (leaching) and toxicity are reduced, the odor nuisance is reduced as the discharge of the active substance into the gas phase is also controlled, photostability and weathering resistance of the active substance are ensured over a longer period of time and originally crystalline active substances are released in amorphous form and over a longer period of time, so that for example leaf penetration is increased.

The invention relates, moreover, to a method for the production of formulations on the basis of organically modified inorganic layered compounds for the controlled, delayed release of active substances, characterized in that
in each case an organically modified layered compound is dispersed in an individual solution of at least one or more active substances in a solvent or a solvent mixture
wherein
either the respective solvents or solvent mixtures differ from one another or
the organically modified inorganic layered compounds differ in at least one modifier or, if the modifiers are the same, these differ in their proportion in the composition or
the unmodified inorganic layered compounds forming the basis of the at least two organically modified inorganic layered compounds are different and
the respective solvent or solvent mixture is then removed,
in each case the resultant powder of organically modified layered compound and active substance is homogenized and
the powders resulting in each case from the different solvent or solvent-mixture preparations are mixed together.

The invention further relates to another method for the production of formulations on the basis of organically modified inorganic layered compounds for the controlled, delayed release of active substances, characterized in that
in each case an organically modified layered compound is dispersed in an individual solution of at least one or more active substances in a solvent or a solvent mixture,
wherein the respective solvents or solvent mixtures differ from one another and
the respective solvent or solvent mixture is then removed,
the powder of organically modified layered compound and active substance resulting in each case is homogenized and
the powders resulting in each case from the different solvent or solvent-mixture preparations are mixed together.

Furthermore, the invention relates to another method for the production of formulations on the basis of organically modified inorganic layered compounds for the controlled, delayed release of active substances, characterized in that
in each case an organically modified layered compound is dispersed in an individual solution of at least one or more active substances in a solvent or a solvent mixture,
wherein the organically modified, inorganic layered compounds each have one or more modifiers and the layered compounds differ in at least one modifier or, if the modifiers are the same, these differ in their proportion in the composition and
the respective solvent or solvent mixture is then removed,
the powder of organically modified layered compound and active substance resulting in each case is homogenized and
the powders resulting in each case from the different solvent or solvent-mixture preparations are mixed together.

The invention relates, moreover, to another method for the production of formulations on the basis of organically modified inorganic layered compounds for the controlled, delayed release of active substances, characterized in that
in each case an organically modified layered compound is dispersed in an individual solution of at least one or more active substances in a solvent or a solvent mixture,
wherein the unmodified inorganic layered compounds forming the basis of the at least two organically modified, inorganic layered compounds are different and
the respective solvent or solvent mixture is then removed,
the powder of organically modified layered compound and active substance resulting in each case is homogenized and
the powders resulting in each case from the different solvent or solvent-mixture preparations are mixed together.

The methods according to the invention are characterized in that the organically modified layered compound is dispersed in a solution of the active substance in one of the solvents stated below or in a solvent mixture. Alternatively, in a first step a dispersion of the modified phyllosilicate and a solution of the active substance in the solvent or solvents are prepared and are then mixed together.

“Different solvents or solvent mixtures” means, in the sense of the invention, solvents that differ fundamentally in their chemical structure or in the case of a mixture in at least one chemical component and/or in their composition.

Furthermore, solvent mixtures are to be understood as those that can have compositions over the entire volume fraction, the only proviso being miscibility.

The solvent is separated from the solid after a certain time of action.

The solvent can preferably be separated by filtration of the solid or by centrifugation and removal of the supernatant. In this advantageous embodiment of the method, excess active substance is largely removed. This can be advantageous for formulations for which an initial release should largely be suppressed.

In another preferred embodiment the formulation can be washed after removing the solvent or solvents, in order to remove excess active substance adsorbed on the outer surface. In this way initial release is suppressed, and only active substance adsorbed on the inner surfaces, which is released later, contributes to the effect.

In an especially preferred embodiment the solvent is removed by distillation or evaporation against a vacuum. An advantage of this method is that no active substance is lost due to the process, as any excess active substance remains adhering to the outer surfaces. Apart from the cost aspect, for certain formulations it may be desirable for a specified increased amount of active substance, relative to the later release, to be released initially, e.g. because in the initial germination phase the germinating seedlings are at their most sensitive to pests.

The residual complex of active substance and organically modified layered compound is for example homogenized by grinding and is mixed with at least one other powder according to the method in accordance with Claims 10-14.

In a preferred embodiment of the method according to the invention, the powder formulation according to the invention can then also be incorporated in other formulations of active substances or multicomponent formulations.

The ratio of active substance to organically modified layered compound is between 0.01 g and 10 g of active substance per gram of layered compound, preferably between 0.1 g and 2 g of active substance per gram of layered compound, especially preferably between 0.2 g and 1 g of active substance per gram of layered compound.

The concentration of the modified layered compound in the solvent is between 0.01 and 50 wt. %, preferably between 0.1 and 30 wt. %, especially preferably between 1 and 10%. Dispersion can be carried out by means of a simple stirrer, shaker, Ultraturrax, ultrasound, high-pressure homogenization or wet grinding.

The time of action is between 10 s and 1 week, preferably between 30 min and 48 h, especially preferably between 1 h and 12 h. Production takes place at temperatures between 0° C. and 200° C., preferably between 0° C. and 100° C., especially preferably between 10° C. and 70° C. under atmospheric pressure and can optionally be carried out under reflux.

Unmodified layered compounds that can be used for the mixtures according to the invention are preferably those of the mineral type montmorillonite, as contained as main constituent in bentonite, or bentonite itself. Moreover, both synthetic and naturally occurring layered compounds can be used, such as the phyllosilicates or clay minerals or clay mineral containing allevardite, amesite, beidellite, bentonite, fluorhectorite, fluorvermiculite, mica, halloysite, hectorite, illite, montmorillonite, muscovite, nontronite, palygorskite, saponite, sepiolite, smectite, stevensite, talc, vermiculite, and synthetic types of talc and the alkali silicates maghemite, magadiite, kenyaite, makatite, silinaite, grumantite, revdite and their hydrated forms and the corresponding crystalline silicas or other inorganic layered compounds such as hydrotalcite, double hydroxides and heteropoly acids, preferably phyllosilicates and double hydroxides.

Different unmodified inorganic layered compounds are to be understood, in the sense of the invention, as those that differ in their chemical composition and/or their structure.

The cation exchange capacities of the anionic layered compounds are between 10 and 260 meq/100 g, preferably between 40 and 200 meq/100 g, especially preferably between 50 and 150 meq/100 g. The anion exchange capacities of the cationic layered compounds (e.g. hydrotalcite, double hydroxides) are between 0.1 and 4.7 meq/g, preferably between 0.5 and 3 meq/g, especially preferably between 0.7 and 2.6 meq/g.

Preferred modifiers of the negatively charged layered compounds are chemical compounds of the alkyl- or arylalkyl-ammonium or -amine or -phosphonium type, whose cationic charges are balanced by the anionic layer charges or by excess anions, for example chloride or bromide ions from the original compounds.

Ammonium compounds are to be understood as those of the formula (NR¹R²R³R⁴)⁺A⁻,

in which
R¹, R², R³ and R⁴, independently of one another, stand in each case for C₁-C₁₈ alkyl, optionally C₂-C₁₈ alkyl interrupted by one or more oxygen atoms, e.g. 1-10 ethylene oxide units, C₆-C₁₂ aryl, C₅-C₁₂ cycloalkyl, wherein the stated residues can in each case be substituted with functional groups, aryl, alkyl, aryloxy, alkyloxy, halogen, heteroatoms and/or heterocycles and/or can have 1-4 double bonds.
R¹, R², R³ and R⁴ can in addition denote hydrogen.
R¹, R², R³ and R⁴ can furthermore denote C₁-C₁₈ alkyloyl (alkylcarbonyl), C₁-C₁₈ alkyloxycarbonyl, C₅-C₁₂ cycloalkylcarbonyl or C₆-C₁₂ aryloyl (arylcarbonyl), wherein the stated residues can in each case be substituted with functional groups, aryl, alkyl, aryloxy, alkyloxy, halogen, heteroatoms and/or heterocycles.

C₁-C₁₈ alkyl optionally substituted with functional groups, aryl, alkyl, aryloxy, alkyloxy, halogen, heteroatoms and/or heterocycles then stands for example for methyl, ethyl, propyl, isopropyl, n-butyl, sec-butyl, tert.-butyl, pentyl, hexyl, heptyl, octyl, 2-ethylhexyl, 2,4,4-trimethylpentyl, decyl, dodecyl, tetradecyl, heptadecyl, octadecyl, 1,1-dimethylpropyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl-1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di-(methoxycarbonyl)-ethyl, 2-methoxyethyl, 2-ethoxyethyl, 2-butoxyethyl, diethoxymethyl, diethoxyethyl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, 2-ethoxyethyl, butylthiomethyl, 2-dodecylthioethyl, 2-phenylthioethyl, 2,2,2-trifluorethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl or 6-ethoxyhexyl and

C₂-C₁₈ alkyl optionally interrupted by one or more oxygen atoms stands for example for 5-hydroxy-3-oxa-pentyl, 8-hydroxy-3,6-dioxa-octyl, 11-hydroxy-3,6,9-trioxa-undecyl, 7-hydroxy-4-oxa-heptyl, 11-hydroxy-4,8-dioxa-undecyl, 15-hydroxy-4,8,12-trioxa-pentadecyl, 9-hydroxy-5-oxa-nonyl, 14-hydroxy-5,10-oxa-tetradecyl, 5-methoxy-3-oxa-pentyl, 8-methoxy-3,6-dioxa-octyl, 11-methoxy-3,6,9-trioxa-undecyl, 7-methoxy-4-oxa-heptyl, 11-methoxy-4,8-dioxa-undecyl, 15-methoxy-4,8,12-trioxa-pentadecyl, 9-methoxy-5-oxanonyl, 14-methoxy-5,10-oxa-tetradecyl, 5-ethoxy-3-oxa-pentyl, 8-ethoxy-3,6-dioxa-octyl, 11-ethoxy-3,6,9-trioxa-undecyl, 7-ethoxy-4-oxa-heptyl, 11-ethoxy-4,8-dioxa-undecyl, 15-ethoxy-4,8,12-trioxa-pentadecyl, 9-ethoxy-5-oxa-nonyl or 14-ethoxy-5,10-oxa-tetradecyl.

functional groups for example for carboxy, carboxamide, hydroxy, di-(C₁-C₄ alkyl)-amino, C₁-C₄ alkyloxycarbonyl, cyano or C₁-C₄ alkyloxy,
C₆-C₁₂ aryl optionally substituted with functional groups, aryl, alkyl, aryloxy, alkyloxy, halogen, heteroatoms and/or heterocycles stands for example for phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, iso-propylphenyl, tert.-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl,
C₅-C₁₂ cycloalkyl optionally substituted with functional groups, aryl, alkyl, aryloxy, alkyloxy, halogen, heteroatoms and/or heterocycles stands for example for cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl and a saturated or unsaturated bicyclic system such as norbornyl or norbornenyl, C₁ to C₄ alkyl stands for example for methyl, ethyl, propyl, isopropyl, n-butyl, sec-butyl or tert.-butyl.

C₁-C₁₈ alkyloyl (alkylcarbonyl) stands for example for acetyl, propionyl, n-butyloyl, sec-butyloyl, tert.-butyloyl, 2-ethylhexylcarbonyl, decanoyl, dodecanoyl, chloroacetyl, trichloroacetyl or trifluoroacetyl.

C₁-C₁₈ alkyloxycarbonyl stands for example for methyloxycarbonyl, ethyloxycarbonyl, propyloxycarbonyl, isopropyloxycarbonyl, n-butyloxycarbonyl, sec-butyloxycarbonyl, tert.-butyloxycarbonyl, hexyloxycarbonyl, 2-ethylhexyloxycarbonyl or benzyloxycarbonyl.

C₅-C₁₂ cycloalkylcarbonyl stands for example for cyclopentylcarbonyl, cyclohexylcarbonyl or cyclododecylcarbonyl.

C₆-C₁₂ aryloyl (arylcarbonyl) stands for example for benzoyl, toluoyl, xyloyl, α-naphthoyl, β-naphthoyl, chlorobenzoyl, dichlorobenzoyl, trichlorobenzoyl or trimethylbenzoyl.

R¹, R², R³ and R⁴, independently of one another, preferably stand for hydrogen, methyl, ethyl, n-butyl, 2-hydroxyethyl, 2-cyanoethyl, 2-(methoxycarbonyl)-ethyl, 2-(ethoxycarbonyl)-ethyl, 2-(n-butoxycarbonyl)-ethyl, dimethylamino, diethylamino and chlorine.

R⁴ preferably stands for methyl, ethyl, n-butyl, 2-hydroxyethyl, 2-cyanoethyl, 2-(methoxycarbonyl)-ethyl, 2-(ethoxycarbonyl)-ethyl, 2-(n-butoxycarbonyl)-ethyl, acetyl, propionyl, t-butyryl, methoxycarbonyl, ethoxycarbonyl or n-butoxycarbonyl.

For phosphinium ions, basically the same substituents apply as for the ammonium ions described in detail.

Especially preferred phosphonium ions corresponding to the formula (PR¹R²R³R⁴)⁺ are those for which, independently of one another

R⁴ stands for acetyl, methyl, ethyl or n-butyl and

R¹, R², and R³ stand for phenyl, phenoxy, ethoxy and n-butoxy.

The ammonium and/or phosphonium ions can also be heterocyclic compounds.

Of these, pyridinium and imidazolium ions are preferred.

The following are quite especially preferred as cations: 1,2-dimethylpyridinium, 1-methyl-2-ethylpyridinium, 1-methyl-2-ethyl-6-methylpyridinium, N-methylpyridinium, 1-butyl-2-methylpyridinium, 1-butyl-2-ethylpyridinium, 1-butyl-2-ethyl-6-methylpyridinium, n-butylpyridinium, 1-butyl-4-methylpyridinium, 1,3-dimethylimidazolium, 1,2,3-trimethylimidazolium, 1-n-butyl-3-methylimidazolium, 1,3,4,5-tetramethylimidazolium, 1,3,4-trimethylimidazolium, 2,3-dimethylimidazolium, 1-butyl-2,3-dimethylimidazolium, 3,4-dimethylimidazolium, 2-ethyl-3,4-dimethylimidazolium, 3-methyl-2-ethylimidazolium, 3-butyl-1-methylimidazolium, 3-butyl-1-ethylimidazolium, 3-butyl-1,2-dimethylimidazolium, 1,3-di-n-butylimidazolium, 3-butyl-1,4,5-trimethylimidazolium, 3-butyl-1,4-dimethylimidazolium, 3-butyl-2-methylimidazolium, 1,3-dibutyl-2-methylimidazolium, 3-butyl-4-methylimidazolium, 3-butyl-2-ethyl-4-methylimidazolium and 3-butyl-2-ethyl imidazolium, 1-methyl-3-octylimidazolium, 1-decyl-3-methylimidazolium.

1-Butyl-4-methylpyridinium, 1-n-butyl-3-methylimidazolium and 1-n-butyl-3-ethylimidazolium are especially preferred.

Basically all anions are conceivable as anions A⁻.

The following are preferred as anions: halides, F⁻, Cl⁻, Br⁻, I⁻, acetate CH₃COO⁻, trifluoroacetate CF₃COO⁻, triflate CF₃SO₃⁻, sulfate SO₄²⁻, hydrogensulfate HSO₄⁻, methylsulfate CH₃OSO₃⁻, ethylsulfate, C₂H₅OSO₃⁻, sulfite SO₃²⁻, hydrogensulfite HSO₃⁻, aluminum chlorides AlCl₄⁻, Al₂Cl₇⁻, Al₃C₁₀⁻, aluminum tetrabromide AlBr₄⁻, nitrite NO₂⁻, nitrate NO₃⁻, copper chloride CuCl₂⁻, phosphate PO₄³⁻, hydrogen phosphate HPO₄²⁻, dihydrogen phosphate H₂PO₄⁻, carbonate CO₃²⁻, hydrogen carbonate HCO₃⁻ or borates, e.g. B(OH)₄⁻.

Preferred modifiers of the positively charged layered compounds (e.g. double hydroxides, hydrotalcites) are carboxylic acids, sulfonic acids or organic sulfates or their salts with alkyl or arylalkyl residues.

Carboxylic acids of the formula (R¹R²R³R⁴)C—COO⁻K⁺ and/or sulfonic acids of the formula (R¹R²R³R⁴)C—SO₃K or alternatively organic sulfates of the formula (R¹R²R³R⁴)C—O—SO₂—O—C(R¹R²R³R⁴) are preferred, in which
R¹, R², R³ and R⁴, independently of one another, stand in each case for C₁-C₁₈ alkyl, optionally C₂-C₁₈ alkyl interrupted by one or more oxygen atoms, e.g. 1-10 ethylene oxide units, C₆-C₁₂ aryl, C₅-C₁₂ cycloalkyl, wherein the stated residues can in each case be substituted with functional groups, aryl, alkyl, aryloxy, alkyloxy, halogen, heteroatoms and/or heterocycles and/or can have 1-4 double bonds.
R¹, R², R³ and R⁴ can moreover stand for hydrogen.
R¹, R², R³ and R⁴ can, furthermore, denote C₁-C₁₈ alkyloyl (alkylcarbonyl), C₁-C₁₈ alkyloxycarbonyl, C₅-C₁₂ cycloalkylcarbonyl or C₆-C₁₂ aryloyl (arylcarbonyl), wherein the stated residues can in each case be substituted with functional groups, aryl, alkyl, aryloxy, alkyloxy, halogen, heteroatoms and/or heterocycles.

C₁-C₁₈ alkyl optionally substituted with functional groups, aryl, alkyl, aryloxy, alkyloxy, halogen, heteroatoms and/or heterocycles then stands for example for methyl, ethyl, propyl, isopropyl, n-butyl, sec-butyl, tert.-butyl, pentyl, hexyl, heptyl, octyl, 2-ethylhexyl, 2,4,4-trimethylpentyl, decyl, dodecyl, tetradecyl, heptadecyl, octadecyl, 1,1-dimethylpropyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl-1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di-(methoxycarbonyl)-ethyl, 2-methoxyethyl, 2-ethoxyethyl, 2-butoxyethyl, diethoxymethyl, diethoxyethyl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, 2-ethoxyethyl, butylthiomethyl, 2-dodecylthioethyl, 2-phenylthioethyl, 2,2,2-trifluorethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl or 6-ethoxyhexyl and

C₂-C₁₈ alkyl optionally interrupted by one or more oxygen atoms, for example for 5-hydroxy-3-oxa-pentyl, 8-hydroxy-3,6-dioxa-octyl, 11-hydroxy-3,6,9-trioxa-undecyl, 7-hydroxy-4-oxa-heptyl, 11-hydroxy-4,8-dioxa-undecyl, 15-hydroxy-4,8,12-trioxa pentadecyl, 9-hydroxy-5-oxa-nonyl, 14-hydroxy-5,10-oxa-tetradecyl, 5-methoxy-3-oxa-pentyl, 8-methoxy-3,6-dioxa-octyl, 11-methoxy-3,6,9-trioxa-undecyl, 7-methoxy-4-oxa-heptyl, 11-methoxy-4,8-dioxa-undecyl, 15-methoxy-4,8,12-trioxa-pentadecyl, 9-methoxy-5-oxanonyl, 14-methoxy-5,10-oxa-tetradecyl, 5-ethoxy-3-oxa-pentyl, 8-ethoxy-3,6-dioxa-octyl, 11-ethoxy-3,6,9-trioxa-undecyl, 7-ethoxy-4-oxa-heptyl, 11-ethoxy-4,8-dioxa-undecyl, 15-ethoxy-4,8,12-trioxa-pentadecyl, 9-ethoxy-5-oxa-nonyl or 14-ethoxy-5,10-oxa-tetradecyl

functional groups for example for carboxy, carboxamide, hydroxy, di-(C₁-C₄ alkyl)-amino, C₁-C₄ alkyloxycarbonyl, cyano or C₁-C₄ alkyloxy,
C₆-C₁₂ aryl optionally substituted with functional groups, aryl, alkyl, aryloxy, alkyloxy, halogen, heteroatoms and/or heterocycles stands for example for phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, iso-propylphenyl, tert.-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl,
C₅-C₁₂ cycloalkyl optionally substituted with functional groups, aryl, alkyl, aryloxy, alkyloxy, halogen, heteroatoms and/or heterocycles stands for example for cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl and a saturated or unsaturated bicyclic system such as norbornyl or norbornenyl,

C₁ to C₄ alkyl stands for example for methyl, ethyl, propyl, isopropyl, n-butyl, sec-butyl or tert.-butyl.

C₁-C₁₈ alkyloyl (alkylcarbonyl) stands for example for acetyl, propionyl, n-butyloyl, sec-butyloyl, tert.-butyloyl, 2-ethylhexylcarbonyl, decanoyl, dodecanoyl, chloroacetyl, trichloroacetyl or trifluoroacetyl.

C₁-C₁₈ alkyloxycarbonyl stands for example for methyloxycarbonyl, ethyloxycarbonyl, propyloxycarbonyl, isopropyloxycarbonyl, n-butyloxycarbonyl, sec-butyloxycarbonyl, tert.-butyloxycarbonyl, hexyloxycarbonyl, 2-ethylhexyloxycarbonyl or benzyloxycarbonyl.

C₅-C₁₂ cycloalkylcarbonyl stands for example for cyclopentylcarbonyl, cyclohexylcarbonyl or cyclododecylcarbonyl.

C₆-C₁₂ aryloyl (arylcarbonyl) stands for example for benzoyl, toluoyl, xyloyl, α-naphthoyl, β-naphthoyl, chlorobenzoyl, dichlorobenzoyl, trichlorobenzoyl or trimethylbenzoyl.

R¹, R², R³ and R⁴, independently of one another, preferably stand for hydrogen, methyl, ethyl, n-butyl, 2-hydroxyethyl, 2-cyanoethyl, 2-(methoxycarbonyl)-ethyl, 2-(ethoxycarbonyl)-ethyl, 2-(n-butoxycarbonyl)-ethyl, dimethylamino, diethylamino and chlorine.

R⁴ preferably stands for methyl, ethyl, n-butyl, 2-hydroxyethyl, 2-cyanoethyl, 2-(methoxycarbonyl)-ethyl, 2-(ethoxycarbonyl)-ethyl, 2-(n-butoxycarbonyl)-ethyl, acetyl, propionyl, t-butyryl, methoxycarbonyl, ethoxycarbonyl or n-butoxycarbonyl.

K stands for any cation, preferably for the ions of the alkali metals or alkaline-earth metals or alternatively for ammonium. Especially preferably, K stands for H, Li⁺, Na⁺, K⁺, Rb⁺, Cs⁺, Mg²⁺, Ca²⁺, Sr²⁺, Ba²⁺, Cu²⁺, Zn²⁺, Fe²⁺, Fe²⁺, Mn²⁺ and NH₄⁺. The cations can be free or can be complexed.

In a preferred embodiment the surface charges of the layered compounds will be compensated between 10 and 200%, preferably between 70 and 130%, especially preferably between 90 and 110%, which corresponds to the degree of coverage of the surface.

The coverage of the surface can—depending on the application—be complete or only partial. In a preferred embodiment of the invention, with partial coverage the unoccupied portion of the inorganic layered compound can still function as a water reservoir or mineral salt donor, and the formulation is generally more wettable with water. In a further preferred embodiment, an almost complete coverage has an advantageous effect in the case of formulations that contain other organic additives, such as adhesives.

Modification of the anionic layered compounds takes place in a manner known by a person skilled in the art, for example by the action of an aqueous solution or of polar organic solutions of ammonium or phosphonium compounds on a dispersion of the unmodified layered compounds (Lagaly, G., Reactions of the clay minerals. In Tonminerale und Tone [clay minerals and clays], Steinkopff Verlag, Darmstadt, 1993). Said ammonium or phosphonium compounds will be used at between 0.1 and 2-fold cation exchange capacity (CEC), preferably between 0.3 and 1.5-fold CEC, especially preferably between 0.4 and 1.2-fold CEC. Moreover, mixtures of at least two of the aforementioned modifiers can be used. The mixtures can be reacted with the layered compound in a one-pot reaction or also sequentially with one modifier in each case in a suitable solvent or solvent mixture one after another, achieving first a partial coverage between 1% and 99% of CEC with one modifier, then further coverage between 1% and 99% of CEC with the next modifier, and so on until there is complete coverage. In this way it is also possible to apply several modifiers.

Modification of the cationic layered compounds is carried out correspondingly, for example by the action of an aqueous solution or solutions in polar organic solvents of carboxylic acids, sulfonic acids or sulfates or their salts on aqueous dispersions or dispersions in polar solvents of the cationic layered compounds or other current methods (Rives, V., Layered Double Hydroxides: present and future, Nova Science Publishers Inc., New York, 2001). The carboxylic acids, sulfonic acids or sulfates are used at between 0.1 and 2-fold anion exchange capacity, preferably between 0.7 and 1.3-fold anion exchange capacity. Moreover, mixtures of at least two of the aforementioned modifiers can be used. The mixtures can be reacted with the layered compound in a one-pot reaction or also, as described above, sequentially with one modifier in each case in a suitable solvent or solvent mixture, one after another.

The layered compounds can be modified specially or it is also possible to use commercially available products of the types Cloisite (Southern Clay Products Inc.), Nanofil (Südchemie), Nanomer (Nanocor Inc.), etc. Correspondingly, Nanofil 15 (distearyldimethylammonium montmorillonite; Südchemie), Nanofil 32 (stearylbenzyldimethylammonium montmorillonite; Südchemie), Nanofil 757 (sodium montmorillonite; Südchemie), Nanofil 784 (aminododecanoic acid montmorillonite; Südchemie), Nanofil 804 (stearyldiethoxyamine montmorillonite), Nanomer 1.30E (octadecylamine montmorillonite; Nanocor, Inc), Nanomer 1.24T (aminododecanoic acid montmorillonite; Nanocor, Inc.) and Nanomer Unmodified Clay (sodium montmorillonite; Nanocor, Inc.) are preferably used.

The active substances for use in the mixtures according to the invention can be, for example, but not conclusively, all substances usually employed for plant treatment, and we may preferably mention fungicides, bactericides, insecticides, acaricides, nematicides, herbicides, plant growth regulators, plant nutrients, and attractants or repellents.

### As Examples of Fungicides we May Mention:

2-Anilino-4-methyl-6-cyclopropyl-pyrimidine; 2′,6′-dibromo-2-methyl-4′-trifluoromethoxy-4-trifluoromethyl-1,3-thiazole-5-carboxanilide; 2,6-dichloro-N-(4-trifluoromethylbenzyl)-benzamide; (E)-2-methoximino-N-methyl-2-(2-phenoxyphenyl)-acetamide; 8-hydroxyquinolinesulfate; methyl (E)-2-{2-[6-(2-cyanophenoxy)-pyrimidin-4-yloxy]-phenyl}-3-methoxyacrylate; methyl-(E) methoximino [alpha-(o-tolyloxy)-o-tolyl]-acetate; 2-phenylphenol (OPP), aldimorph, ampropylfos, anilazin, azaconazole, benalaxyl, benodanil, benomyl, binapacryl, biphenyl, bitertanol, blasticidine-S, bromuconazole, bupirimate, buthiobate, calcium polysulfide, captafol, captan, carbendazim, carboxin, quinomethionate, chloroneb, chloropierin, chlorothalonil, chlozolinate, cufraneb, cymoxanil, cyproconazole, cyprofuram, carpropamide, dichlorophen, diclobutrazole, dichlofluanid, diclomezin, dicloran, diethofencarb, difenoconazole, dimethirimol, dimethomorph, diniconazole, dinocap, diphenylamine, dipyrithion, ditalimfos, dithianon, dodine, drazoxolon, edifenphos, epoxyconazole, ethirimol, etridiazole, fenarimol, fenbuconazole, fenfuram, fenitropan, fenpiclonil, fenpropidin, fenpropimorph, fentin acetate, fentin hydroxide, ferbam, ferimzone, fluazinam, fludioxonil, fluoromide, fluquinconazole, flusilazole, flusulfamide, flutolanil, flutriafol, folpet, fosethyl-aluminum, phthalides, fuberidazole, furalaxyl, furmecyclox, fenhexamide, guazatine, hexachlorobenzene, hexaconazole, hymexazole, imiazalil, imibenconazole, iminoctadin, iprobenfos (IBP), iprodion, isoprothiolan, iprovalicarb, kasugamycin, copper preparations, such as: copper hydroxide, copper naphthenate, copper oxychloride, copper sulfate, copper oxide, oxine-copper and Bordeaux mixture, mancopper, mancozeb, maneb, mepanipyrim, mepronil, metalaxyl, metconazole, methasulfocarb, methfuroxam, metiram, metsulfovax, myclobutanil, nickel dimethyldithiocarbamate, nitrothal-isopropyl, nuarimol, ofurace, oxadixyl, oxamocarb, oxycarboxin, pefurazoate, penconazole, pencycuron, phosdiphen, pimaricin, piperalin, polyoxin, probenazole, prochloraz, procymidon, propamocarb, propiconazole, propineb, pyrazophos, pyrifenox, pyrimethanil, pyroquilon, quintozen (PCNB), quinoxyfen, sulfur and sulfur preparations, spiroxamines, tebuconazole, teclophthalam, tecnazen, tetraconazole, thiabendazole, thicyofen, thiophanate-methyl, thiram, tolclophos-methyl, tolylfluanide, triadimefon, triadimenol, triazoxide, trichlamide, tricyclazole, tridemorph, triflumizol, triforin, triticonazole, trifloxystrobin, validamycin A, vinclozolin, zineb, ziram, and 2-[2-(1-chloro-cyclopropyl)-3-(2 chlorophenyl)-2-hydroxypropyl]-2,4-dihydro-[1.2.4]-triazole-3-thione.

### As Examples of Bactericides we May Mention:

Bronopol, dichlorophen, nitrapyrin, nickel-dimethyldithiocarbamate, kasugamycin, octhilinon, furancarboxylic acid, oxytetracycline, probenazole, streptomycin, teclophthalam, copper sulfate and other copper preparations.

### As Examples of Insecticides, Acaricides and Nematicides we May Mention:

Abamectin, acephate, acetamiprid, acrinathrin, alanycarb, aldicarb, alphamethrin, amitraz, avermectin, AZ 60541, azadirachtin, azinphos A, azinphos M, azocyclotin, Bacillus thuringiensis, 4-bromo-2-(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, BPMC, brofenprox, bromophos A, bufencarb, buprofezin, butocarboxin, butylpyridaben, cadusafos, carbaryl, carbofuran, carbophenothion, carbosulfan, cartap, chloethocarb, chlorethoxyfos, chlorfenvinphos, chlorfluazuron, chlormephos, N-[(6-chloro-3-pyridinyl)-methyl]-N′-cyano-N-methyl-ethanimidamide, chlorpyrifos, chlorpyrifos M, cis-resmethrin, clocythrin, clofentezin, clothianidin, cyanophos, cycloprothrin, cyfluthrin, cyhalothrin, cyhexatin, cypermethrin, cyromazin, deltamethrin, demeton-M, demeton-S, demeton-5-methyl, diafenthiuron, diazinon, dichlofenthion, dichlorvos, dicliphos, dicrotophos, diethion, diflubenzuron, dimethoate, dimethylvinphos, dioxathion, disulfoton, emamectin, esfenvalerate, ethiofencarb, ethion, ethofenprox, ethoprophos, etrimphos, fenamiphos, fenazaquin, fenbutatin oxide, fenitrothion, phenobucarb, phenothiocarb, phenoxycarb, fenpropathrin, fenpyrad, fenpyroximate, fenthion, fenvalerate, fipronil, fluazuron, flucycloxuron, flucythrinate, flufenoxuron, flufenprox, fluvalinate, fonophos, formothion, fosthiazate, fubfenprox, furathiocarb, HCH, heptenophos, hexaflumuron, hexythiazox, imidacloprid, iprobenfos, isazophos, isofenphos, isoprocarb, isoxathion, ivermectin, lambda-cyhalothrin, lufenuron, malathion, mecarbam, mevinphos, mesulfenphos, metaldehyde, methacrifos, methamidophos, methidathion, methiocarb, methomyl, metolcarb, milbemectin, monocrotophos, moxidectin, naled, NC 184, nitenpyram, omethoate, oxamyl, oxydemethon M, oxydeprofos, parathion A, parathion M, permethrin, phenthoate, phorate, phosalon, phosmet, phosphamidon, phoxim, pirimicarb, pirimiphos M, pirimiphos A, profenophos, promecarb, propaphos, propoxur, prothiophos, prothoate, pymetrozin, pyrachlophos, pyridaphenthion, pyresmethrin, pyrethrum, pyridaben, pyrimidifen, pyriproxifen, quinalphos, salithion, sebufos, silafluofen, sulfotep, sulprofos, tebufenozide, tebufenpyrad, tebupirimiphos, teflubenzuron, tefluthrin, temephos, terbam, terbufos, tetrachlorvinphos, thiacloprid, thiafenox, thiamethoxam, thiodicarb, thiofanox, thiomethon, thionazin, thuringiensin, tralomethrin, transfluthrin, triarathen, triazophos, triazuron, trichlorfon, triflumuron, trimethacarb, vamidothion, XMC, xylylcarb, zetamethrin.

Especially preferably, the powder formulations according to the invention contain imidacloprid, thiacloprid, thiamethoxam, acetamiprid, clothianidin, betacyfluthrin, cypermethrin, transfluthrin, lambda-cyhalothrin and/or azinphosmethyl.

### As Examples of Herbicides we May Mention:

Anilides, such as diflufenican and propanil; arylcarboxylic acids, such as dichloropicolinic acid, dicamba and picloram; aryloxyalkanoic acids, such as 2,4-D, 2,4-DB, 2,4-DP, fluoroxypyr, MCPA, MCPP and triclopyr; aryloxy-phenoxy-alkanoic acid esters, such as diclofop-methyl, phenoxaprop-ethyl, fluazifop-butyl, haloxyfop-methyl and quizalofop-ethyl; azinones, such as chloridazon and norflurazon; carbamates, such as chlorpropham, desmedipham, phenmedipham and propham; chloroacetanilides, such as alachlor, acetochlor, butachlor, metazachlor, metolachlor, pretilachlor and propachlor; dinitroanilines, such as oryzalin, pendimethalin and trifluralin; diphenyl ethers, such as acifluorfen, bifenox, fluoroglycofen, fomesafen, halosafen, lactofen and oxyfluorfen; ureas, such as chlortoluron, diuron, fluometuron, isoproturon, linuron and methabenzthiazuron; hydroxylamines, such as alloxidim, clethodim, cycloxydim, sethoxydim and tralkoxydim; imidazolinone such as imazethapyr, imazamethabenz, imazapyr and imazaquin; nitriles, such as bromoxynil, dichlobenil and ioxynil; oxyacetamides, such as mefenacet; sulfonyl ureas, such as amidosulfuron, bensulfuron-methyl, chlorimuronethyl, chlorsulfuron, cinosulfuron, metsulfuron-methyl, nicosulfuron, primisulfuron, pyrazosulfuron-ethyl, thifensulfuron-methyl, triasulfuron and tribenuron-methyl; thiolcarbamates, such as butylates, cycloates, diallates, EPTC, esprocarb, molinate, prosulfocarb, thiobencarb and triallate; triazines, such as atrazine, cyanazine, simazine, simetryne, terbutryne and terbutylazine; triazinones, such as hexazinone, metamitron and metribuzin; miscellaneous compounds, for example aminotriazole, benfuresate, bentazone, cinmethylin, clomazone, clopyralid, difenzoquat, dithiopyr, ethofumesate, fluorochloridone, glufosinate, glyphosate, isoxaben, pyridate, quinchlorac, quinmerac, sulphosate and tridiphane. Others that may be mentioned are 4-amino-N-(1,1-dimethylethyl)-4,5-dihydro-3-(1-methylethyl)-5-oxo-1H-1,2,4-triazole-1-carboxamide and benzoic acid, 2-((((4,5-dihydro-4-methyl-5-oxo-3-propoxy-1H-1,2,4-triazol-1-yl)carbonyl)amino)sulfonyl)-methyl ester.

Especially preferably, the powder formulations according to the invention contain propoxycarbazone-sodium, flucarbazone-sodium, amicarbazone dichlobenil and/or phenyluracils.

As examples of plant growth regulators we may mention chlorocholine chloride and ethephon.

As examples of plant nutrients we may mention usual inorganic or organic fertilizers for supplying plants with macro and/or micro nutrients.

As examples of repellents we may mention diethyl-tolylamide, ethylhexanediol, 1-piperidinecarboxylic acid 2-(2-hydroxyethyl)-1-methylpropyl ester (Bayrepel®) and butopyronoxyl.

In a further preferred embodiment, pharmacological, veterinary-medical and cosmetic actives, such as aromatics or odorants, or material-protection active substances can be formulated, for which a linearized release profile with continuous release of active substance is desirable.

All customary solvents or solvent mixtures in the conceivable mixture proportions can be used as solvents for the active substances in the method according to the invention or as dispersing agents for the layered compounds. These solvents or solvent mixtures cause swelling of the organically modified phyllosilicates to a varying extent. Possible solvents are:

hydrocarbons, pure or mixtures thereof (C₅-C₁₈), for example n-pentane, n-hexane, n-heptane, n-octane, petroleum ether.

Halogenated hydrocarbons, for example mono-, di-, tri-, tetra-chlorocarbon, preferably dichloromethane and chloroform, ethers—such as diethyl ether, glycol dimether, esters such as propylene glycol-monomethylether-acetate, dibutyl adipate, ethyl acetate, hexyl acetate, heptyl acetate, tri-n-butyl citrate, diethyl phthalate and di-n-butyl phthalate, ketones—such as acetone, methyl-isobutyl ketone and cylohexanone, alcohols—such as methanol, ethanol, n- and i-propanol, n- and i-butanol, n- and i-amyl alcohol, benzyl alcohol and 1-methoxy-2-propanol or combinations thereof, and amides—such as dimethylformamide or dimethylacetamide, furthermore, strongly polar solvents such as DMSO, in addition cyclic compounds, such as N-methyl-pyrrolidone, N-octyl-pyrrolidone, N-dodecyl pyrrolidone, N-octyl-caprolactam, N-dodecyl-caprolactam and γ-butyrolactone or cyclic mono- or diethers such as THF and dioxan, nitriles such as acetonitrile, also aromatic hydrocarbons such as xylene, toluene, benzene, nitrophenol. In addition, water can be used as diluent.

These same solvents can also be used for further formulation of the powder formulations according to the invention.

The powder formulations according to the invention can be used as such or after addition of further formulation aids for application of agrochemical active substances in plant protection both in agriculture and forestry, and in horticulture. As formulation aids, consideration may be given to all usual components that can be used in plant treatment agents, for example dyes, wetting agents, dispersants, emulsifiers, antifoaming agents, preservatives, components that delay drying, antifreezing agents, secondary thickening agents, solvents and, in the case of manufacture of seed-treatment solutions, also adhesives or polymeric binders.

As dyes that can be used for further preparation of the powders according to the invention as plant treatment agents, consideration may be given to all the usual dyes for such purposes. Both pigments that are sparingly soluble in water and water-soluble dyes can be used. As examples, we may mention the dyes known by the designations Rhodamine B, C.I. Pigment Red 112 and C.I. Solvent Red 1.

As wetting agents that can be used for formulation of the powders according to the invention, consideration may be given to all the usual wetting-promoting substances for formulation of agrochemical active substances. Alkylnaphthalene-sulfonates, such as diisopropyl or diisobutyl-naphthalene-sulfonates, can preferably be used.

As dispersants and/or emulsifiers that can be used for formulation of the powders according to the invention, consideration may be given to all the usual nonionic, anionic and cationic dispersants for formulation of agrochemical active substances. Nonionic or anionic dispersants or mixtures of nonionic or anionic dispersants can preferably be used. As suitable nonionic dispersants we may mention in particular ethylene oxide-propylene oxide copolymers, alkylphenolpolyglycol ethers and tristyrylphenolpolyglycol ethers and their phosphatized or sulfatized derivatives. Suitable anionic dispersants are in particular lignin sulfonates, polyacrylic acid salts and arylsulfonate-formaldehyde condensates.

As antifoaming agents that can be used for formulation of the powders according to the invention, consideration may be given to all the usual foam inhibiting substances for formulation of agrochemical active substances. Silicone antifoaming agents and magnesium stearate can preferably be used.

As preservatives that can be used for formulation of the powders according to the invention, consideration may be given to all the usual substances for such purposes for formulation of agrochemical active substances. As examples we may mention dichlorophen and benzyl alcohol-hemiformal.

As components that delay drying and as antifreezing agents that can be used for formulation of the powders according to the invention, consideration may be given to all substances that can be used for such purposes in agrochemicals. Consideration may preferably be given to polyhydric alcohols, such as glycerol, ethanediol, propanediol and polyethylene glycols of various molecular weights.

As secondary thickening agents that can be used for formulation of the powders according to the invention, consideration may be given to all substances that can be used for such purposes in agrochemicals. Consideration may preferably be given to cellulose derivatives, acrylic acid derivatives, xanthan, and highly-dispersed silica.

Another object of the invention is the use of the powder formulations according to the invention as seed dressings.

For formulation of the powders according to the invention as seed dressings, adhesives are then also used. All the usual binders that can be used in seed-treatment solutions may be considered.

We may preferably mention polyvinyl pyrrolidone, polyvinyl acetate, polyvinyl alcohol, biodegradable polymers, such as polylactides, collagen, gelatin, cellulose and cellulose derivatives, starch and its derivatives and tylose.

Dispersions of biodegradable polyester-polyurethane-polyureas in water are also especially preferred as adhesives. Such dispersions are known (cf. WO 01-17347).

The powder formulations according to the invention can be used in practice, as such or also after mixing with other formulation aids and/or plant treatment agents and optionally after further dilution with water. Application is by the usual methods, for example by scattering, pouring, sprinkling or spraying.

A further object of the invention is the use of the powder formulations according to the invention in spray application. The adsorption of crystalline active substances in the amorphous state prevents recrystallization and thus promotes leaf penetration.

The invention is explained in more detail below on the basis of the following examples, but is not restricted to these.

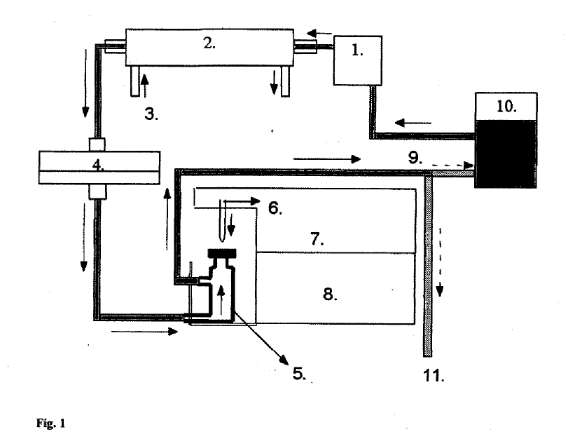

FIG. 1 shows a schematic diagram of the setup of the measuring apparatus used for the release tests (carried out according to Example 10),

FIG. 2 shows the plot of the amount released of active substance imidachloprid F as percentage of formulations of active substances prepared in various solvents from Example 2 (b. water, c. acetone, d. N-heptane, e. ethanol) and mixture thereof (a.) from Example 6 with pure imidachloprid as reference (f. crystalline imidacloprid) plotted against time t,

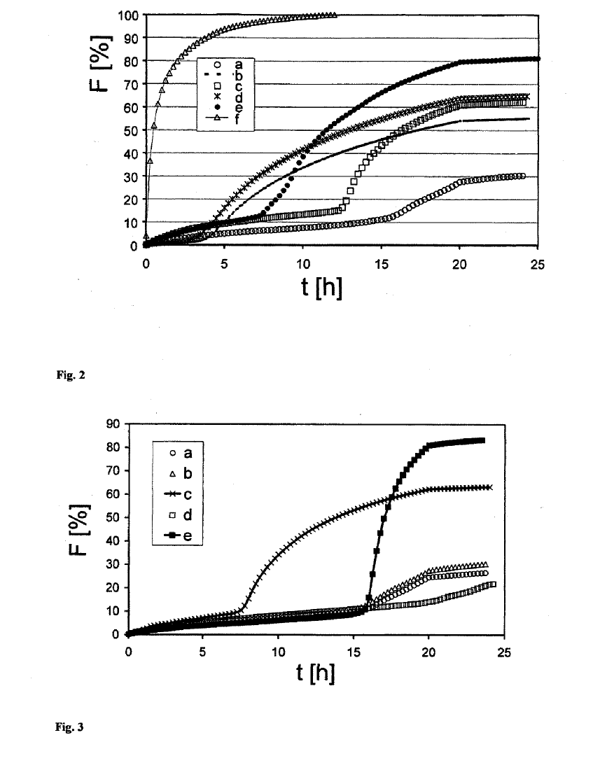

FIG. 3 shows the plot of the amount released of active substance imidachloprid F as percentage of formulations of active substances prepared in various ethanol/toluene mixture ratios from Example 4 plotted against time t (a. 100% ethanol, b. 80% ethanol, c. 50% ethanol, d. 20% ethanol, e. 0% ethanol),

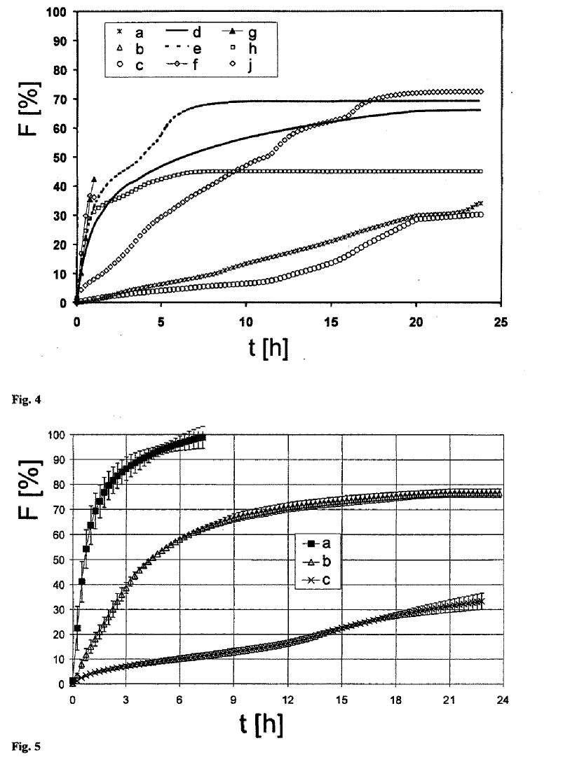

FIG. 4 shows the plot of the amount released of active substance imidachloprid F as percentage of formulations of active substances prepared from various modified phyllosilicates in ethanol from Example 5 (a. Nanofil 15, b. Nanofil 784, c. Nanomer 130E, d. Nanofil 32, e. Nanofil 804, f. Nanomer sodium form, g. Nanofil 757, h. Nanomer 124T) and mixture thereof from Example 7 (j. mixture) plotted against time t and

FIG. 5 shows the plot of the amount released of active substance imidachloprid F as percentage from rice dressings prepared according to Example 8 from active substance formulations in ethanol prepared according to Example 1 and Example 2 (b. rice dressings phyllosilicate formulation from ethanol) and from rice dressings prepared according to Example 9 from a mixture of active substance formulations according to Example 6 (c. rice dressings phyllosilicate formulation mixture) with pure imidachloprid rice dressings (a. rice dressings imidacloprid) as reference, plotted against time t.

### EXAMPLES

The phyllosilicates used were Nanofil 15 (distearyldimethylammonium montmorillonite; Südchemie), Nanofil 32 (stearylbenzyldimethylammonium montmorillonite; Südchemie), Nanofil 757 (sodium montmorillonite; Südchemie), Nanofil 784 (aminododecanoic acid montmorillonite; Südchemie), Nanofil 804 (stearyldiethoxyamine montmorillonite), Nanomer 1.30E (octadecylamine montmorillonite; Nanocor, Inc.), Nanomer 1.24T (aminododecanoic acid montmorillonite; Nanocor, Inc.) and Nanomer Unmodified Clay (sodium montmorillonite; Nanocor, Inc.).

### Example 1

### Preparation of Organic Phyllosilicate Dispersions

For the preparation of an approx. 1 wt. % dispersion of organically modified phyllosilicate in a solvent, 5 g Nanomer 1.30E (Nanocore Inc.) was dispersed in 500 g solvent using an Ultraturrax stirrer (TuraxT25 S25N-18G) at 20 500 rev/min. The dispersions were shaken overnight (approx. 15 h) on a laboratory shaker (150 rpm, RT). Dispersions were prepared correspondingly in the following solvents: water, n-heptane, DMSO, acetone, ethanol, toluene.

### Example 2

### Formulation of Active Substances by Means of Phyllosilicate-Solvent Dispersions

200 mg IMIDACLOPRID was then added to the dispersions from Example 1. The dispersions were shaken overnight on the shaker at RT and then evaporated to dryness in the rotary evaporator at 45° C., dried thoroughly in a vacuum drying cabinet at 55° C. (25 mbar, 5-7 days) and ground in a vibratory mill at 30 Hz for 5 min in 25 ml PE beakers with two ZrO₂ mill balls (d=10 mm).

The release behavior was analyzed as in Example 10. The results are shown in FIG. 2 (b. water, c. acetone, d. N-heptane, e. ethanol and mixture thereof (a.) from Example 6).

### Example 3

### Preparation of Organic Phyllosilicate Dispersions in Solvent Mixtures

Formulations were prepared in solvent mixtures of ethanol and toluene in different mass fractions. The solvents were mixed in the following ethanol/toluene ratios:

| | | |
|---|---|---|
| w (EtOH) = 0 | 396 | g toluene + 0 g ethanol |
| w (EtOH) = 0.2 | 316.8 | g toluene + 179.2 g ethanol |
| W (EtOH) = 0.5 | 198.0 | g toluene + 198.0 g ethanol |
| w (EtOH) = 0.8 | 79.2 | g toluene + 316.8 g ethanol |
| w (EtOH) = 1 | 0 | g toluene + 396 g ethanol |

4 g of Nanomer 1.30E was dispersed in each solution (1 min in the Ultraturrax at 20500 rev/min). The dispersions were shaken overnight on the laboratory shaker (150 rpm, RT).

### Example 4

### Formulations of Active Substances Using Phyllosilicate/Solvent Mixture Dispersions

160 mg IMIDACLOPRID was then added to the dispersions from Example 3 and pulverulent formulations were prepared as in Example 2. The release behavior was analyzed as in Example 10. The results are shown in FIG. 3 (a. 100% ethanol, b. 80% ethanol, c. 50% ethanol, d. 20% ethanol, e. 0% ethanol).

### Example 5

### Formulations of Active Substances Based on Variously Modified Phyllosilicates

Powder formulations based on variously modified phyllosilicates were prepared as in Example 2. Several 500 mL glass bottles were each filled with 396 g ethanol. 4 g of phyllosilicate was dispersed in each solution (1 min in the Ultraturrax at 20500 rev/min). The phyllosilicates used were Nanofil 15 (distearyldimethylammonium montmorillonite; Südchemie), Nanofil 32 (stearylbenzyldimethylammonium montmorillonite; Südchemie), Nanofil 757 (sodium montmorillonite; Südchemie), Nanofil 784 (aminododecanoic acid montmorillonite; Südchemie), Nanofil 804 (stearyldiethoxyamine montmorillonite), Nanomer 1.30E (octadecylamine montmorillonite; Nanocor, Inc), Nanomer 1.24T (aminododecanoic acid montmorillonite; Nanocor, Inc.) and Nanomer Unmodified Clay (sodium montmorillonite; Nanocor, Inc.).

The dispersions were shaken overnight on the laboratory shaker (150 rpm, RT), then 160 mg IMIDACLOPRID was added in each case, dispersed (30 s in the Ultraturrax at 20500 rev/min) and the dispersions were shaken overnight on the laboratory shaker (150 rpm, RT). Then the dispersions were concentrated in the rotary evaporator at 45° C. and then dried in the vacuum drying cabinet (at <10 mbar & 40° C.). The dried samples were ground in a vibratory mill at 30 Hz for 5 min with two ZrO₂ mill balls (d=10 mm).

The release behavior was analyzed as in Example 10. The results are shown in FIG. 4 (a. Nanofil 15, b. Nanofil 784, c. Nanomer 130E, d. Nanofil 32, e. Nanofil 804, f. Nanomer sodium form, g. Nanofil 757, h. Nanomer 124T; j. mixture)

### Example 6

### Mixtures from Example 2

Powder formulations from Example 2 were mixed. For this, in each case 300 mg of the pulverulent formulations originally prepared from acetone, DMSO, ethanol and n-heptane, and 30 mg of the pulverulent formulation prepared from water were mixed in an agate mortar, and the release was measured as in Example 10. The results are shown in FIG. 2 (b. water, c. acetone, d. N-heptane, e. ethanol and mixture thereof (a.)).

### Example 7

### Mixtures from Example 5

Powder formulations from example were mixed. For this, in each case approx. 250 mg of imidacloprid-clay powder formulations (4 wt. % IMIDACLOPRID per g clay) based on Nanomer 1.30 E, Nanofil 15, Nanofil 32, Nanofil 757 were mixed, homogenized in an agate mortar and mixture weighing approx. 260 mg was transferred to each release cell. The release was measured as in Example 10. The results are shown in FIG. 4 (a. Nanofil 15, b. Nanofil 784, c. Nanomer 130E, d. Nanofil 32, e. Nanofil 804, f. Nanomer sodium form, g. Nanofil 757, h. Nanomer 124T; j. mixture) as “mixture”.

### Example 8

### Release from a Rice Dressing (Clay Formulation from Ethanol)

1 g of Nanomer 130.E-imidacloprid mixture (10 wt. % imidacloprid per g clay, prepared as in Example 1 or Example 2 from ethanol) was dispersed in 6 g of water/ethanol mixture (1:1) for 10 min using a magnetic stirrer. Then 0.052 g of the adhesive Impranil DLN Dispersion W 50 (approx. 50 wt. %, from Bayer Material Science) and 0.5112 g of the dye dispersion Levanyl Red BB-LF (1 wt. %, from LanXess) were added. This dispersion was shaken vigorously. The result was a highly viscous suspension.

3.7 g of this suspension was mixed with 12 g rice in a small beaker using a spatula, until it could be seen that the red suspension adhered to the grains of rice. Then the treated rice was stored for approx. 42 h in a drying cabinet at 40° C. Next, approx. 3.1 g of the dried rice dressing was put in each release cell.

As a comparative sample, in addition a rice dressing was prepared as stated above, containing pure imidacloprid instead of the phyllosilicate formulation. 93.5 mg of imidacloprid was added instead of the Nanomer 130.E-imidacloprid mixture. In the release tests, approx. 2.3 g of this comparative formulation was used per release cell, to obtain the same amount of imidacloprid as in the phyllosilicate formulation.

There was a marked delay in release from the phyllosilicate formulation.

Release was measured as in Example 10. The results are shown in FIG. 5 (a. rice dressing imidacloprid, b. rice dressings phyllosilicate formulation from ethanol, c. rice dressings phyllosilicate formulation mixture) as “rice dressings phyllosilicate formulation from ethanol”.

### Example 9

### Release from a Rice Dressing (Mixture)

As in Example 8, a rice dressing was prepared starting from a mixture of phyllosilicate-imidacloprid powders from Example 6 (prepared from the solvents acetone, DMSO, ethanol, toluene, n-hexane). Release was measured as in Example 10. The results are shown in FIG. 5 (a. rice dressing imidacloprid, b. rice dressings phyllosilicate formulation from ethanol, c. rice dressings phyllosilicate formulation mixture) as “rice dressings phyllosilicate formulation mixture”. For comparison, a rice dressing was prepared as in Example 8 with pure IMIDACLOPRID; the results are shown in FIG. 5 (a. rice dressing imidacloprid, b. rice dressings phyllosilicate formulation from ethanol, c. rice dressings phyllosilicate formulation mixture) as “rice dressings imidacloprid”.

### Example 10

### Release Measurements: Description of Flow Measurements

Approx. 260 mg of pulverulent formulation was applied uniformly in a flow cell on a glass-fiber prefilter (Millipore), with a cellulose ester filter (0.1 μm, diameter 47 mm, from Millipore) underneath, and it was sealed (see FIG. 1, No. 4). For comparability of the samples, the same imidacloprid/clay ratios and the same absolute amounts of imidacloprid were used in the release measurements. The release cells were made of polycarbonate, the hoses were of the Tygon LFL type (diameter 2.2 mm; wall thickness 0.84 mm). A stream of deionized water (Millipore) at 4 ml/min flowed through the release cells in flow-through mode (see FIG. 1, No. 9+11).

At intervals of 5 min for single determinations or 15 min for parallel triple determinations, 5 μl sample volumes were taken automatically (see FIG. 1, No. 6+7) and injected into the HPLC (Hewlett Packard 1050; see FIG. 1, No. 8). Separation was carried out in a Lichrospher® 100RP8 separation column (5 μm; Lycrocart® 125-4). Acetonitrile/water was used as the solvent system, operating a gradient (10% ACN/90% H₂O pH 2; after 2 min 90% ACN/10% H₂O pH 2) at a flow of 1.5 ml/min. Detection used a variable wavelength detector at a wavelength of 282 nm. The analysis time was 3.5 min with 1.5 min posttime. The concentrations released were found from calibration curves and were plotted cumulatively against time. The release between two measurement points was assumed to be linear. This assumption was verified by extraction of the residual formulation after discontinuing the flow measurements by a mass balance (estimation of whether a burst between two measurement points falsifies the mass balance).

### REFERENCE SYMBOLS

1. Telab® BF 414 membrane pump
2. Julabo cryostat
3. Thermostat circuit (RT)
4. Release flow cell with sample
5. Flow vessel (VA steel, own design)
6. HPLC injection needle
7. HPLC autosampler
8. HPLC with variable wavelength detector
9. Water circuit (recycling mode)
10. Storage container for the release agent (e.g. deionized water)
11. Decrease (flow-through mode)

1. Powder formulations for controlled, delayed release of active substances comprising:
at least two organically modified, inorganic layered compounds, each comprising at least one active substance and
optionally additives
wherein said powder formulations can be obtained by a method of production in which
in each case an organically modified layered compound is dispersed in a separate solution of one or more active substances in a solvent or a solvent mixture,
wherein
either the respective solvents or solvent mixtures differ from one another or
the organically modified inorganic layered compounds differ in at least one modifier or, if the modifiers are the same, these differ in their proportion in the composition or
the unmodified inorganic layered compounds forming the basis of the at least two organically modified, inorganic layered compounds are different and
the respective solvent or solvent mixture is then removed,
the powder of organically modified layered compound and active substance resulting in each case is homogenized and
the powders resulting in each case from the different solvent or solvent-mixture preparations are mixed together.

2. The powder formulations for controlled, delayed release of active substances as claimed in claim 1 comprising:
at least two organically modified, inorganic layered compounds, each comprising at least one active substance and
optionally additives
wherein said powder formulations can be obtained by a method of production in which
in each case an organically modified layered compound is dispersed in a separate solution from at least one active substance in a solvent or a solvent mixture,
wherein the respective solvents or solvent mixtures differ from one another and
the respective solvent or solvent mixture is then removed,
the powder of organically modified layered compound and active substance resulting in each case is homogenized and
the powders resulting in each case from the different solvent or solvent-mixture preparations are mixed together.

3. The powder formulations for controlled, delayed release of active substances as claimed in claim 1 comprising:
at least two organically modified, inorganic layered compounds, each comprising at least one active substance and
optionally additives
wherein said powder formulations can be obtained by a method of production in which
in each case an organically modified layered compound is dispersed in a separate solution from one or more active substances in a solvent or a solvent mixture,
wherein the organically modified, inorganic layered compounds each have one or more modifiers and the layered compounds differ in at least one modifier or, if the modifiers are the same, these differ in their proportion in the composition and
the respective solvent or solvent mixture is then removed,
the powder of organically modified layered compound and active substance resulting in each case is homogenized and
the powders resulting in each case from the different solvent or solvent-mixture preparations are mixed together.

4. The powder formulations for controlled, delayed release of active substances as claimed in claim 1, comprising:
at least two organically modified, inorganic layered compounds, each comprising at least one active substance and
optionally additives
wherein said powder formulations can be obtained by a method of production in which
in each case an organically modified layered compound is dispersed in a separate solution from one or more active substances in a solvent or a solvent mixture,
wherein the unmodified inorganic layer compounds forming the basis of the at least two organically modified, inorganic layered compounds are different and
the respective solvent or solvent mixture is then removed,
the powder of organically modified layered compound and active substance resulting in each case is homogenized and
the powders resulting in each case from the different solvent or solvent-mixture preparations are mixed together

5. Powder formulations, which are mixtures of the powder formulations as claimed in claim 1.

6. The powder formulations as claimed in claim 1, wherein the layered compounds are organically modified phyllosilicates or organically modified double hydroxides.

7. The powder formulations as claimed in claim 1, wherein the active substances are agrochemical active substances.

8. The powder formulations as claimed in claim 1, wherein the anionic inorganic layered compounds are those with a cation exchange capacity between 10 and 260 meq/100 g.

9. The powder formulations as claimed in claim 1, wherein the cationic inorganic layered compounds are those with an anion exchange capacity between 0.1 and 4.7 meq/g.

10. A method for the production of formulations on the basis of organically modified inorganic layered compounds for the controlled, delayed release of active substances, comprising the steps of:
in each case an organically modified layered compound is dispersed in an individual solution of at least one or more active substances in a solvent or a solvent mixture
wherein
either the respective solvents or solvent mixtures differ from one another or
the organically modified inorganic layered compounds differ in at least one modifier or, if the modifiers are the same, these differ in their proportion in the composition or
the unmodified inorganic layered compounds forming the basis of the at least two organically modified, inorganic layered compounds are different and
the respective solvent or solvent mixture is then removed,
the powder of organically modified layered compound and active substance resulting in each case is homogenized and
the powders resulting in each case from the different solvent or solvent-mixture preparations are mixed together.

11. A method for the production of formulations on the basis of the organically modified inorganic layered compounds for the controlled, delayed release of active substances as claimed in claim 10, wherein
in each case an organically modified layered compound is dispersed in an individual solution of at least one or more active substances in a solvent or a solvent mixture,
wherein the respective solvents or solvent mixtures differ from one another and
the respective solvent or solvent mixture is then removed,
the powder of organically modified layered compound and active substance resulting in each case is homogenized and
the powders resulting in each case from the different solvent or solvent-mixture preparations are mixed together.

12. A method for the production of formulations on the basis of the organically modified inorganic layered compounds for the controlled, delayed release of active substances as claimed in claim 10, wherein
in each case an organically modified layered compound is dispersed in an individual solution of at least one or more active substances in a solvent or a solvent mixture,
wherein the organically modified, inorganic layered compounds each have one or more modifiers and the layered compounds differ in at least one modifier or, if the modifiers are the same, these differ in their proportion in the composition and
the respective solvent or solvent mixture is then removed,
the powder of organically modified layered compound and active substance resulting in each case is homogenized and
the powders resulting in each case from the different solvent or solvent-mixture preparations are mixed together.

13. A method for the production of formulations on the basis of the organically modified inorganic layered compounds for the controlled, delayed release of active substances as claimed in claim 10, wherein
in each case an organically modified layered compound is dispersed in an individual solution of at least one or more active substances in a solvent or a solvent mixture,
wherein the unmodified inorganic layer compounds forming the basis of the at least two organically modified, inorganic layered compounds are different and
the respective solvent or solvent mixture is then removed,
the powder of organically modified layered compound and active substance resulting in each case is homogenized and
the powders resulting in each case from the different solvent or solvent-mixture preparations are mixed together.

14. The method as claimed in claim 10, wherein the ratio of active substance and organically modified layered compound is between 0.01 g and 10 g of active substance per gram of layered compound.

15. A seed dressing or a sprayable mixture comprising a powder formulation as claimed in claim 1.

16. An agrochemical, material-protection, pharmaceutical, veterinary-medical or cosmetic formulation comprising a powder formulation as claimed in claim 1.
